(19) United States
(12) Patent Application Publication
(10) Pub. No.: US 2011/0236560 A1
(43) Pub. Date: Sep. 29, 2011

(54) FOOD SUPPLEMENTATION WITH PHENOLIC ANTIOXIDANTS IN VINEGAR

(76) Inventor: Daniel Perlman, Arlington, MA (US)

(21) Appl. No.: 12/703,832

(22) Filed: Feb. 11, 2010

(51) Int. Cl. A23L 1/30; C07C 39/00

(52) U.S. Cl. 426/648

(57) ABSTRACT

A food or beverage composition suitable for human consumption is described which includes an aqueous acetic acid or acetate salt solution that has been supplemented with phenolic antioxidants, and may also contain an omega-3 fatty acid-containing supplementation oil in which a supplementation oil includes a blend of an omega-3 fatty acid-containing enriching oil that has been combined and diluted with an oxidative stabilization oil, such as a low linoleic acid/high oleic acid-containing oxidative stabilization oil or a high saturated fat/low linoleic acid-containing fat. The combination of aqueous acetic acid and phenolic antioxidants can result in reduction or elimination of astringency due to the presence of the phenolic antioxidants, and can further reduce the levels of damaging free radicals resulting from cooking processes. In addition, the acetic acid can provide chemical stabilization of the phenolic antioxidants.

### RELATED APPLICATIONS

Not applicable

### FIELD OF THE INVENTION

The present invention relates to liquid and non-liquid foods supplemented with phenolic antioxidants and also containing aqueous acetate ion, including food such as salad dressings, vinegar-containing condiments, and the like, resulting in reduction in astringency of the phenolic antioxidants. If the conditions are acidic, the phenolic antioxidants are beneficially stabilized against degradation. The foods can also include omega-3 fatty acids stabilized in certain oils that prevent the food from developing a fishy flavor.

### BACKGROUND OF THE INVENTION

The following discussion is provided solely to assist the understanding of the reader, and does not constitute an admission that any of the information discussed or references cited constitute prior art to the present invention.

It has been recognized in recent years that the diets of many individuals are deficient in one or more ways. As a result, it has become common to supplement commonly consumed foods with one or more functional components to make the supplemented components more widely available in the diets of many people. Common examples include vitamin supplementation such as the addition of vitamins A and D in milk, folic acid in flour, niacin and iron in bread, calcium in non-dairy beverages, and the like.

There has also been recognition of the health benefits derived from consuming a number of additional micronutrient components found in a variety of foods. For example, foods rich in plant-derived sterols and stanols, fruit and vegetable-derived phenolic antioxidants, and omega-3 fatty acids are considered beneficial. A number of such components have also been made available as nutraceutical dietary supplements, and in some cases, as food additives.

These developments have led to a large interest in functional foods, and a large number of patent applications in this general area have been submitted. For example, McCleary et al., US Pat Publ 20050002992 mentions a variety of different categories of health functions for supplemented foods and a plethora of combinations of foods or beverages and additives which might be constructed.

As noted above, recognition of the health benefits of phenolic antioxidants has led to recommendations that individuals should eat more fruits and vegetables that are rich in such compounds. Unfortunately, many if not most people consume insufficient amounts of foods rich in phenolic antioxidants, and fail to obtain the accompanying health benefits. One possible solution is to increase the number of commonly ingested foods that contain significant amounts of phenolic antioxidants, thereby facilitating ingestion of effective amounts of the antioxidants. A major limitation of this approach is that many fruits and vegetables that are good sources of phenolic antioxidants are costly and only seasonally available. Another limitation is that many phenolic antioxidant compounds tend to be chemically unstable in many processed food environments. This particularly poses a problem for foods that must be stored for a significant period of time, e.g., foods that must have a shelf life of weeks or months.

While there are many different bioactive phenolic antioxidants, many health benefits have been attributed to the dietary consumption of the group of water-soluble phenolic antioxidants known as the proanthocyanidins. A partial list of health conditions that have been reported to benefit from regular ingestion of proanthocyanidins are as follows: heart disease and atherosclerosis, pancreatic inflammation, cancer cell proliferation, kidney, lung and heart cell damage (e.g., damage caused by chemotherapeutic drug treatments). Related phenolic antioxidants have been shown to beneficially modulate or control blood platelet aggregation, LDL oxidation, endothelial dysfunction, rheumatoid arthritis and leukemia cell propagation. A bibliography that encompasses much of the recent research (years 2000-2005) involving phenolic antioxidants and their role in controlling disease is provided in the book, Muscadine Medicine by Hartle, Greenspan and Hargrove (2005) ISBN Number 1-4116-4397-6. More specifically, with regard to the health benefits provided by proanthocyanidins in the diet, several informative review articles are available at, for example, http://www.blackwell-synergy.com/doi/pdf/10.1111/j.1469-8137.2004.01217.x and at http://repositories.cdlib.org/cgi/viewcontent.cgi?article=1045&context=uclabiolchem/nutritionnoteworthy.

Phenolic compounds have been included in a variety of food products, including some in which acidic conditions were present, e.g., as described in a number of patent applications from the Unilever Corporation. For example, in Graff & Hrncirik, WO 2007/048471, an aqueous fluid wine extract was added to a variety of food products, including fruit juice products (p. 9), dairy products (p. 10), frozen confectionary products (p. 10), nutrition bars (p. 11), and food emulsions/spreads (p. 12-13). Particularly, in connection with the spreads, a pH of 4.2-6.0 was mentioned (p. 13, line 4). Similarly in a set of four US patent application filed by Unilever on the same day and all entitled Composition Comprising Polyphenol, single phenolic compounds were used in food products substantially the same as those indicated above. The application publications are: Draijer et al., US Pat Appl Publ 20090011103 (coumaric acid); Draijer et al., US Pat Appl Publ 20090012183 (trans-resveratrol); Draijer et al., US Pat Appl Publ 20090010993 (kaempferol); and Draijer et al., US Pat Appl Publ 20090012156 (isorhamnetin). Reference to a pH of 4.2-6.0 (the same as in WO 2007/048471) was made in each application in paragraphs 41, 38, 43, and 37 respectively. Zhang, US Pat Appl Publ 20090017183 (assigned to the same company, Unilever, as the Graff and Draijer applications mentioned above) described compositions in which a plant-derived acid such as gallic acid or p-coumaric acid (among others) was used to in combination with tea catechins; the pH of the resulting beverage solution was generally in the range of 2.5 to about 6.0 (paragraph 34).

Also, as mentioned above, omega-3 fatty acids have been recognized as essential and have been provided as dietary supplements and, in some case, as food additives. Omega-3 fatty acids constitute a family of polyunsaturated fatty acids that are recognized as providing a wide range of health benefits when consumed as a regular part of the human diet. The most well known omega-3 fatty acids include alpha-linolenic acid (ALA) that is found in soybean oil, canola oil and flaxseed oil, as well as docosahexaenoic acid (DHA), and eicosapentaenoic (EPA) commonly found in fish oil and other marine oils. All of these fatty acids contain multiple carbon-carbon double bonds including one double bond in the omega-3 or third position inward from the distal end of the fatty acid chain that is attached at its opposite end by an ester linkage to the glycerol backbone of the triglyceride molecule.

While the human body is not capable of synthesizing omega-3 fatty acids from other nutrients, it is able to convert some of the dietary alpha-linolenic acid that is 18 carbons in length, to the longer 20 and 22 carbon chain EPA (20:5 n-3) and DHA (22:6 n-3) molecules. Both the omega-3 fatty acids and the omega-6 fatty acid, linoleic acid (18:2n-6), are termed “essential nutrients” because they are largely obtained from foods rather than synthesized by the body.

In recent years, the U.S. FDA allowed a “qualified health claim” to be made with regard to the dietary consumption of EPA and DHA, stating that “supportive but not conclusive research shows that consumption of EPA and DHA omega-3 fatty acids may reduce the risk of coronary heart disease.”

A variety of medical conditions have been reported to be ameliorated by regular dietary consumption of EPA and DHA. Some of these conditions include improvement in blood circulation, control of heart arrhythmias, beneficial control of clot formation, reduction in blood pressure, beneficial reduction of blood triglyceride levels, reduced risk of primary and secondary heart attacks, and improvements covering wide range of inflammatory diseases including rheumatoid arthritis. Some research has suggested that fish oil may limit the risk of thrombotic and ischemic stroke as well, while beneficially reducing the amount of LDL cholesterol oxidation that occurs in the bloodstream and that may contribute to atherogenesis.

Some studies indicate that the incidence of certain forms of cancer including prostate, breast and colon is reduced by substantial dietary intake of omega-3 fatty acids. Still other research has suggested that omega-3 fatty acids may ameliorate conditions of psychological depression and anxiety.

While maximum safe levels of EPA and DHA have not been established, it is believed that daily intake of 4 grams EPA and 2 grams DHA are not excessive. Since many typical fish oils contain approximately 30% by weight EPA+DHA, it is likely that consuming up to 20 grams per day of fish oil would result in no adverse health effects. Many people consume between one and six 1 g capsules of fish oil per day, providing between approximately 300-1800 mg of EPA and DHA. While these levels may be desirable goals for many health-conscious individuals, it is believed that making even a fraction of these levels available to the general public by supplementing conventional foods will result in a significant public health benefit.

### SUMMARY OF THE INVENTION

The present invention addresses significant difficulties encountered in supplementing foods with phenolic antioxidants by providing food compositions to which are added both phenolic antioxidants and acetate ion. Use of the added acetate, preferably in the form of acetic acid significantly reduces the astringency experienced by individuals from consuming many types of phenolic antioxidants. Addition of acetate ion in an acidic environment (e.g., by adding acetic acid) also beneficially stabilizes the phenolic antioxidants against chemical degradation which would otherwise take place.

As indicated above, many of the phenolic antioxidants contribute a highly unpleasant astringent taste when added to foods and beverages in appreciable quantities. The present invention overcomes these problems by incorporating the phenolic antioxidants into an acetic acid-containing solution or liquid suspension containing acetic acid. The acetic acid solution or suspension is most often a vinegar. When suitably combined, the acetic acid surprisingly substantially reduces organoleptic astringency experienced with many solubilized phenolic antioxidants. In turn, this reduced astringency allows supplementation of foods with significantly higher levels of beneficial antioxidants while still allowing foods to remain fully palatable. In addition, the invention concerns the use of edible phenolic antioxidants, preferably obtained from fruits and/or vegetables, to pre-treat or condition foods before cooking in order to reduce the levels of damaging free radicals and deleterious reaction products generated during food cooking processes, e.g., during grilling. In this application of the invention, the presence of acetic acid in the pre-treatment composition, e.g., in a marinating sauce, can provide as many as four benefits simultaneously. The acetic acid reduces excessive phenolic astringency, imparts a desirable flavor, acts (as an acid) to tenderize meat, fish and poultry, and chemically stabilizes the phenolic antioxidants.

Thus, a first aspect of the invention concerns a method for supplementing a food composition with phenolic antioxidants by mixing an edible aqueous acetate solution (or the molar equivalent of acetate solution) with at least one edible phenolic antioxidant preparation, e.g., enriched or purified phenolic antioxidants, thereby providing a phenolic antioxidant-supplemented food composition. In many cases, the method will also include combining the acetic acid solution and phenolic antioxidant preparation with one or more other components, providing a food composition which is a prepared food composition. Advantageously, the acetate is introduced in or to an acidic environment, e.g., added as an aqueous acetic acid solution or acidified after addition of an acetate salt. Preferably the phenolic antioxidants are added at a water-astringent level, e.g., at least 1.5, 1.7, 2, 3, 4, or 5 times a level which would be excessively astringent in water without the acetate (e.g., acetic acid).

In particular embodiments, the prepared food composition is a salad dressing, a barbecue sauce, a cooking sauce, a mayonnaise, a mustard; the phenolic antioxidant-supplemented food composition is substantially non-astringent or at least the astringency is substantially reduced over the astringency which would be present in the absence of the acetic acid.

In some embodiments, the phenolic antioxidants are added as purified phenolic antioxidants, such as a water-soluble powdered extract selected from the group consisting of grape seed extract and Camellia sinensis extract; the colloidal added phenolic antioxidants are or include plant matter flour, e.g., one or more of fruit seed flour (e.g., grape seed, raspberry seed, blueberry seed, pomegranate seed), fruit skin flour, and plant leaf flour (e.g., Camellia sinensis flour), and combinations thereof (e.g., Camellia sinensis flour and grape seed flour).

In advantageous embodiments, the acetic acid solution contains 0.2 to 10 percent by weight acetic acid, e.g., 0.2 to 2, 0.2 to 8, 0.2 to 7, 0.2 to 6, 0.2 to 5, 0.2 to 4, 0.2 to 3, 0.2 to 2, 0.5 to 10, 0.5 to 7, 0.5 to 5, 0.5 to 4, 0.5 to 3, 0.5 to 2, 1 to 10, 1 to 7, 1 to 5, or 1 to 3 percent by weight acetic acid (or a molar equivalent of acetate ion, which may be acidified in the solution); the acetate ion and phenolic antioxidants are in an aqueous solution with a pH of 2.0 to 5.5, 2.0 to 5.0, 2.0 to 4.5, 2.0 to 4.0, 2.5 to 5.5, 2.5 to 5.0, 2.5 to 4.5, 2.5 to 4.0, 3.0 to 5.5, 3.0 to 5.0, 3.0 to 4.5, or 3.0 to 4.0; the weight ratio of supplementary phenolic antioxidants or total phenolic antioxidants to acetic acid in the supplemented acetic acid solution is in a range of 0.01 to 10, e.g., 0.01 to 5 0.01 to 2, 0.01 to 1, 0.01 to 0.5, 0.01 to 0.2, 0.01 to 0.1, 0.01 to 0.05, 0.01 to 0.02, 0.1 to 10, 0.1 to 5, 0.1 to 2, 0.1 to 1.0, 0.1 to 0.5, 0.5 to 10, 0.5 to 5, 0.5 to 2 (or a ratio of acetate ion provided by the molar equivalent of acetate ion, which may be acidified in the solution); the acetic acid (or acetate) solution is supplemented with phenolic antioxidants where the total level of phenolic antioxidants in the aqueous acetic acid portion, or alternatively the level of exogenously added phenolic antioxidants in the aqueous acetic acid (or acetate) portion is from 0.10 to 2.00%, 0.10 to 1.50%, 0.10 to 1.00%, 0.20 to 2.00%, 0.20 to 1.50%, 0.20 to 1.00%, 0.50 to 2.00%, 0.50 to 1.50%, 0.50 to 1.00%, 2.00 to 3.00%, 2.00 to 4.00%, 2.00 to 5.00%, 2.00 to 6.00%, or 4.00 to 6.00% by weight of the aqueous acetic acid (or acetate) portion; the prepared food includes supplemented antioxidants at a level providing at least 10, 15, 20, 25, 30, 40, 60, 70, 80, or 100 mg (GAE equivalents) per serving of the prepared food.

In particular embodiments, the phenolic antioxidants are stabilized sufficiently that the level of phenolic antioxidants present in the food composition after a period of one month at 20 degrees C. is at least 1.5, 2, 3, 4, 5, 7, or 10 times the level of phenolic antioxidants present in the same food composition prepared with water instead of the aqueous acetic acid solution.

It was also found that the present combinations of phenolic antioxidants and acetate ion (e.g., from vinegar) can be combined with stabilized omega-3 supplementation without interfering with either supplementation. Thus, in certain embodiments, the method also includes combining the aqueous acetate (e.g., acetic acid) and phenolic antioxidant preparation with a stabilized omega-3 fatty acid-containing edible oil, e.g., an omega-3-rich oil within an oxidative stabilization oil containing a limited amount of linoleic acid as described in Perlman U.S. Pat. No. 7,344,747 and in Perlman, U.S. patent application Ser. No. 12/143,729, filed Jun. 20, 2008 and/or Perlman U.S. patent application Ser. No. 12/276,447, filed Nov. 24, 2008, each of which is incorporated herein by reference in its entirety; the resulting prepared food product is a salad dressing, a mayonnaise, a mustard sauce, a cooking sauce, or a barbecue sauce.

It has been found that dietary consumption of omega-3 fatty acids is desirable in order to provide certain health benefits. Advantageously, such omega-3 fatty acids can be provided in the oil component of a food product. Thus, the present method can also include incorporating omega-3 fatty acids in a fat/oil component of a food product, where the omega-3 fatty acids (and preferably other polyunsaturated fatty acids) are diluted in a stabilizing oil so that the oxidation rate of those fatty acids is reduced, preferably sufficiently reduced to provide a significantly increased product life. In most cases, this is accomplished by diluting the omega-3 fatty acids or oils high in such omega-3 fatty acids in an oxidative stabilization oil prior to blending the oils with other components of a food product or food product component. Creation of the artificial blend of omega-3 fatty acid-containing oil and an oxidative stabilization oil is itself counterintuitive, because for common prior uses of omega-3 fatty acid-containing oils, e.g., as food supplements or nutraceuticals, it would be undesirable on both an effective concentration basis and on a transport cost basis to dilute the omega-3 oil in a bulk oil. Discovery of the effectiveness of the approach using a blend of an omega-3 fatty acid-rich oil with an oxidative stabilization oil further led to the realization that particular types of single oils and other oil blends could also be used to provide omega-3 fatty acid supplementation in milk and other oil-water products.

Thus, the method includes combining an edible omega-3 fatty acid-containing supplementation oil suitable for human consumption in a food product, where the supplementation oil contains docosahexaenoic acid (DHA) and/or eicosapentaenoic (EPA) fatty acids, highly preferably at a combined level sufficient to provide at least 10 mg of DHA plus EPA per normal serving of the food composition. In many cases, the supplementation oil contains one part by weight of an omega-3 enriching oil containing DHA and/or EPA, that has been combined and diluted with at least one part by weight of an oxidative stabilization oil, however, many other ratios can be used in particular cases, e.g., depending on the compositions of each of the oils.

In particular embodiments, the rate of oxidation of the DHA and EPA fatty acids is reduced to no more than 0.80, 0.70, 0.50, 0.30, 0.20, 0.10, 0.05, 0.02, 0.01, or 0.005 of the rate of oxidation of an equal quantity of the EPA/DHA fatty acid-containing omega-3 enriching oil homogenized or otherwise blended in droplet (preferably microdroplet) form into the aqueous acetic acid solution without having been combined and diluted with the oxidative stabilization oil, or reduced to within a range which is defined by taking any two different just specified values as the endpoints of the range; the rate of oxidation of the EPA/DHA fatty acids added per normal serving of the food product via the supplementation oil is reduced between 2- and 400-fold, 2 and 100-fold, 4- and 400-fold, 4- and 200-fold, 4- and 100-fold, 4- and 50-fold, 6- and 400-fold, 6- and 200-fold, 6- and 100-fold, 6- and 50-fold, 10- and 400-fold, 10- and 200-fold, 10- and 100-fold, 10- and 50-fold, 50- and 400-fold, or 100- and 400-fold, or even more compared to the rate of oxidation of the same quantity of the EPA/DHA fatty acid-containing enriching oil homogenized into the aqueous suspension without having been first combined and diluted with the oxidative stabilization oil.

In certain embodiments, the oxidative stabilization oil contains no more than 20, 15, 12, 11, 10, 9, or 8% by weight of polyunsaturated fatty acids, or specifically of linoleic acid; the oxidative stabilization oil contains at least 60, 65, 70, 75, 80, 85, or 90% by weight of monounsaturated fatty acids and/or saturated fatty acids; the oxidative stabilization oil contains at least 60, 65, 70, 75, 80, or 85% by weight of oleic acid; the oxidative stabilization oil contains no more than 20, 15, 12, 11, 10, 9, or 8% by weight of polyunsaturated fatty acids, or specifically of linoleic acid and at least 60, 65, 70, 75, 80, or 85% by weight of monounsaturated fatty acids (e.g., contains the specified percentage of oleic acid); the oxidative stabilization oil is a low linoleic acid and high oleic acid oil (commonly a vegetable oil), e.g., a low linoleic acid and high oleic acid sunflower seed oil; the oxidative stabilization oil is high oleic vegetable oil, e.g., high oleic sunflower oil, high oleic safflower oil, high oleic canola oil, and/or high oleic soybean oil; the oxidative stabilization oil is corn oil, sunflower oil, safflower oil, soybean oil, cottonseed oil, canola oil, peanut oil, palm fat, coconut fat, cocoa butter, palm oil, palm olein, palm kernel oil, milkfat, a milkfat fraction, and/or animal fat; the oxidative stabilization oil is solid at normal storage temperature for the composition, e.g., solid at 25, 22, 20, 18, 15, 12, 10, 8, 7, 6, 5, 4, or 3 degrees Celsius or cooler; the oxidative stabilization oil contains no more than 15, 12, 11, 10, 9, 8, 7, 6, 5, 4, 3, 2, or 1% by weight ALA and/or no more than 2, 1.5, 1, 0.7, 0.5, 0.2, or 0.1% EPA+DHA; the oxidative stabilization oil satisfies the ALA and/or EPA+DHA levels just specified and also satisfies any of the limitations specified for an oxidative stabilization oil as specified in this paragraph or otherwise specified herein.

Also in certain embodiments, the EPA/DHA fatty acid-containing enriching oil includes at least 15, 20, 25, 30, 35, 40, 45, 50, 55, or 60% (or even higher) by weight of the long chain polyunsaturated fatty acids EPA, DHA, and combinations thereof, or contains EPA, DHA, or a combination thereof in a range of between 15 and 60%, 20 and 60%, 25 and 60%, 30 and 60%, or 40 and 60%; the EPA/DHA fatty acid-containing enriching oil is or includes fish oil, e.g., at least 50, 60, 70, 80, or 90% by weight fish oil; the EPA/DHA fatty acid-containing enriching oil is or includes algae oil; the structural isomeric arrangement of EPA and/or DHA fatty acids contained within the triglyceride molecules of the EPA/DHA fatty acid-containing enriching oil have not been altered from their native structural arrangement; the EPA and/or DHA fatty acids contained within the triglyceride molecules of said EPA/DHA fatty acid-containing enriching oil have been interesterified, and the average number of the EPA and/or DHA fatty acids per triglyceride molecule has been increased.

In particular embodiments, one part by weight of an EPA/DHA fatty acid-containing enriching oil has been combined and diluted with approximately (or at least approximately) 0.5, 0.7, 1, 2, 3, 4, 5, 7, 10, 12, 15, 17, or 20 parts by weight of an oxidative stabilization oil, e.g. a low linoleic acid/high oleic acid-containing oxidative stabilization oil, or with between 0.5 and 5 parts, 1 and 5 parts, 2 and 5 parts, 2 and 10 parts, 2 and 20 parts, 5 and 10 parts, 5 and 20 parts, 10 and 15 parts or 10 and 20 parts by weight of an oxidative stabilization oil.

For some embodiments, between 5 and 500 mg, 10 and 200 mg, 10 and 100 mg, 50 and 500 mg, 50 and 200 mg, 50 and 100 mg, 100 and 500 mg, or 100 and 200 mg of EPA or DHA fatty acids or a combination of both are added per normal serving of the food product.

In certain embodiments in which there are separate oil and water phases (e.g., as an emulsion) in the composition, the oil phase includes at least one oil soluble and water insoluble antioxidant, highly preferably at a concentration effective to provide significant antioxidant protection to unsaturated fatty acids (and especially to polyunsaturated fatty acids, including omega-3 fatty acids) in that oil phase. Such antioxidants may, for example, include BHA and/or BHT (e.g., at levels up to 100 ppm by weight of either or each) and/or ascorbyl palmitate (also referred to as vitamin C palmitate, e.g., at levels of up 1000 ppm by weight).

Thus, in particular embodiments, the oil phase includes 10 to 100, 20 to 100, or 50 to 100 ppm of BHA and/or BHT, and/or 20 to 1000, 50 to 1000, 100 to 1000, 50 to 500, 100 to 500, 200 to 700, or 200 to 500 ppm ascorbyl palmitate; the oil phase includes effective amounts of at least two, three, or four different approved oil soluble/water insoluble antioxidants; the oil phase includes at least a 3, 4, 5, 7, 10, 15, or 20-fold dilution of an omega-3 fatty acid enriching oil in an oxidative stabilization oil and at least one oil soluble/water insoluble antioxidant, preferably effective to reduce the oxidation rate of polyunsaturated fatty acids to no more than 0.9, 0.8, 0.7, 0.5, 0.3, 0.2, or 0.1 of the rate in the absence of the antioxidant(s); the oil phase includes vitamin E (e.g., at a level of 200 to 2000 ppm by weight or even higher) and at least one other oil soluble/water insoluble antioxidant, e.g., an antioxidant(s) as described for other embodiments herein.

In particular embodiments, the supplementation oil is a single oil or an oil blend which contains EPA and/or DHA at levels such that the combination of the two is no more than 20% by weight of that oil, and preferably no more than 17, 15, 12, 10, 8, 7, 6, or 5% by weight of the supplementation oil and/or the supplementation oil contains ALA, preferably at a level of no more than 30% by weight, or more preferably at a level of no more than 25, 20, 15, or 10% by weight; such supplementation oil may, for example be a blend of an omega-3 fatty acid-enriching oil and an oxidative stabilization oil, a blend of two more oils of which none by itself is an oxidative stabilization oil, or a single oil selected or designed to provide the desired omega-3 fatty acid levels. In particular embodiments, the levels of other polyunsaturated fatty acids or specifically of linoleic acid in the supplementation oil is limited, e.g., such that the non-omega-3 polyunsaturated fatty acids or specifically linoleic acid constitute no more than 20, 15, 12, 11, 10, 9, 8, 7, 6, 5, or 4% by weight of the supplementation oil, and/or the supplementation oil contains at least 30, 40, 50, 60, 65, 70, 75, 80, or 85% by weight of oleic acid or combination of monounsaturated fatty acids or at least 30, 40, 50, 60, 65, 70, 75, 80, or 85% by weight of oleic acid or combination of monounsaturated fatty acids and 3 to 25, 5 to 25, 10 to 25, 3 to 15, 3 to 10, 5 to 15, or 5 to 10% by weight of saturated fatty acids (preferably where the monounsaturated fatty acid to saturated fatty acid ratio is at least 1.5, 2, 3, 5, 7, or 10.

A related aspect of the invention concerns a method for reducing free radicals and/or deleterious compounds formed in a food composition during cooking by contacting the food composition with a solution or suspension which is or includes at least one edible phenolic antioxidant preparation, e.g., enriched or purified phenolic antioxidants, mixed with an edible aqueous acetic acid solution (or other aqueous acetate solution, which may be acidified). During cooking, the presence of the phenolic antioxidants reduces the amount of free radicals or deleterious free radical reaction products present compared to the levels which are present in the absence of the phenolic antioxidant and acetic acid combination.

In particular embodiment, the deleterious compounds are or include polycyclic aromatic hydrocarbons (PAHs) and/or acrylamides.

In particular embodiments, e.g., depending on nature of the food composition, the phenolic antioxidant and acetic acid solution or suspension may be added to the food composition such that it is distributed throughout the food composition or may be coated on the surface of the food composition, e.g., as a barbecue sauce, marinade, or other surface coating cooking sauce.

Also in particular embodiments, the type(s) of phenolic antioxidants and/or the composition of the phenolic antioxidant and acetic acid solution or suspension is as described for the aspect above or otherwise described for the present invention.

The food composition may be a variety of different foods, but in certain embodiments is a meat product, e.g., a ground meat product such as hamburger or a sliced meat product such as steak or cutlets, or ribs.

Similarly, in another related aspect, the invention concerns a food product suitable for human consumption that includes a combination of aqueous acetic acid solution (or molar equivalent acetate ion, which may be acidified) and supplementary phenolic antioxidants dissolved in the aqueous acetic acid solution.

In particular embodiments, the phenolic antioxidant-supplemented aqueous acetic acid solution (or other acetate solution, which may be acidified), the aqueous acetic acid solution, the phenolic antioxidants, and/or the food product are as specified for the preceding aspect, or otherwise described herein for the present invention.

In particular embodiments, the aqueous acetic acid solution has been supplemented and blended (e.g., homogenized) with an omega-3 fatty acid-containing supplementation oil, where the omega-2 fatty acid-containing supplementation oil includes one part by weight of an alpha-linolenic fatty acid-containing omega-3 fatty acid enriching oil (e.g., fish oil and/or flax seed oil), that has been combined and diluted with at least one part by weight of an oxidative stabilization oil.

In particular embodiments, the reduction of the rate of oxidation, the type and/or amount of oxidative stabilization oil, the ratio of the enriching oil and the stabilization oil, the type of food product are as described for embodiments of the preceding aspect.

Additional embodiments will be apparent from the Detailed Description and from the claims.

### DETAILED DESCRIPTION OF THE PREFERRED EMBODIMENTS

In recent years, the medical community has become increasingly aware of the importance of regularly consuming substantial amounts of phenolic antioxidants as a part of the diet. Such phenolic antioxidants are generally provided by a variety of different dietary plant materials. Unfortunately, the diets of many people are deficient in such plant materials, such that those individuals ingest inadequate amounts of phenolic antioxidants. As a result, it has become desirable to supplement various foods with phenolic antioxidants. As significant limitation to the addition of substantial quantities of many phenolic antioxidants to foods and beverages is that supplementation at even moderately high levels commonly produces an objectionable astringent taste or sensation, such that many individuals find the phenolic antioxidant supplemented food or beverage composition unpalatable. Added to this problem is the chemical instability of those antioxidants, leading to undesirably rapid degradation under normal food preparation and/or storage conditions.

Thus, the present invention provides an advantageous method for supplementing certain foods and beverages with substantial levels of phenolic antioxidants while reducing or eliminating the astringency difficulty by combining the phenolic antioxidants with aqueous acetate ion, most often added in the form of acetic acid.

Furthermore, the present method for supplementing foods and beverages with phenolic antioxidants can be advantageously combined with a method for stabilized supplementation of foods and beverages with omega-3 fatty acids which beneficially can be ingested as a regular part of the human diet. The addition of fish oil, algae oil, and/or flaxseed oil as omega-3 enriching oils to foods can help ensure people will regularly consume omega-3 fatty acids. However, a difficulty with such additions has been that the fish oils or other omega-3 fatty acid-containing oils can relatively rapidly develop a disagreeably fishy odor/flavor due to degradation products when present together with an aqueous phase.

As part of this invention, it was found that the phenolic antioxidant (e.g., as acetic acid) with acetate supplementation can be used in combination with addition of stabilized omega-3 fatty acids without reducing the effectiveness of either supplementation component. Therefore, the present invention also concerns the joint supplementation of foods or beverages which have both aqueous and oil phases with phenolic antioxidants as indicated above and with stabilized omega-3 fatty acids. In this way the phenolic antioxidants have their astringency taste reduced, and the chemical stability of omega-3 fatty acids is significantly extended. This helps maintain the functional benefits of the supplements while ensuring that the flavor of the enriched food product will not be unacceptably compromised by oxidation of omega-3 fatty acids. As indicated above, phenolic antioxidants can also be stabilized against degradation by using acetic acid or other edible acidifier.

Omega-3 fatty acid supplementation applicable to the present invention is described in Perlman U.S. Pat. No. 7,344,747 and in Perlman, U.S. patent application Ser. No. 12/143,729, filed Jun. 20, 2008 and/or Perlman U.S. patent application Ser. No. 12/276,447, filed Nov. 24, 2008, each of which is incorporated herein by reference in its entirety.

The present phenolic antioxidant and/or omega-3 fatty acid supplementation method is applicable, for example, to many salad dressings, mayonnaise preparations, mustard sauces (commonly mustard condiment sauces), and barbecue sauces, among others.

### Production of Phenolic Antioxidant Supplemented Aqueous Acetate (e.g., Acetic Acid)

Supplementation of aqueous acetate solutions, such as acetic acid solutions (e.g., vinegar), with phenolic antioxidants is conveniently accomplished. In most cases, the phenolic antioxidants are soluble in aqueous solutions, and will readily solubilize when a powdered phenolic antioxidant preparation is thoroughly mixed with the aqueous solution. While the phenolic antioxidants can be first dissolved in aqueous solution which does not contain appreciable acetic acid (or acetate ion from an acetate salt), with the acetic acid (or acetate) added subsequently, it is preferable if the phenolic antioxidants are dissolved in aqueous acetic acid (or acetate) solution or at least added at substantially the same time as the acetic acid (or acetate). If necessary, mild heating can be used to accelerate the process. The acetate salts such as the sodium, potassium, and calcium acetate salts as well as acetic acid are also highly soluble in water and can be simply dissolved and distributed with stirring. If an acetate salt is used, preferably the solution or the food is acidified with an edible acid.

The phenolic antioxidant-containing aqueous acetic acid (or acetate) solution may be made before the solution is combined with other food components, e.g., oil, or the aqueous acetic acid may be combined with one or more other components of the food prior to addition of the phenolic antioxidants.

As indicated above, the general population benefits from regularly consuming more fruit and vegetables rich in phenolic antioxidants, and processed foods fortified with phenolic antioxidants that are part of a healthy diet. Phenolic antioxidant molecular diversity and broader health functionality can be provided by dietary consumption of a variety of sources of phenolic antioxidants. In principle, such diversity can permit multiple health conditions to be treated with regular dietary intake of diverse phenolic antioxidants rather than a single antioxidant compound. An increase of 25%, 50%, and preferably 100% or more in phenolic antioxidant content over the endogenous level present in a processed food via admixture of exogenous antioxidants can be achieved for a minimal cost, i.e., approximately 0.1-1 cent per serving.

In recent years, the scientific literature has suggested that different species of phenolic (commonly polyphenolic) molecules can exhibit different biochemical properties and provide a range of health benefits when consumed regularly in the human diet. Thus, it is believed that a combining of phenolic antioxidants, e.g., from grape seeds and teas for example, may provide greater health benefits than from either individually. It is contemplated that in some instances, the antioxidants from tea and grape seed be combined, e.g., in approximately equal proportions based upon their phenolic antioxidant activities as measured in ORAC or GAE units.

A diversity and balance between glycosylated and aglycone phenols may also be desirable. For example, with acai berries, Del Pozo-Insfran et al., J. Agric. Chem. (2006) 54(4):1222-1229 demonstrated that the glycosylated forms of polyphenolic acids and flavanols were more potent in affecting leukemia cell proliferation and cell death in culture than aglycone forms. Thus, in some cases the present invention incorporates both glycosylated and aglycone phenols (e.g., in a balanced combination), preferably with a diversity of chemical species as discussed above.

The biological functionality of these phenolic antioxidants as anti-inflammatory agents and agents to reduce both harmful oxidation of LDL cholesterol and platelet aggregation in the bloodstream, can be enhanced by the further addition of a triglyceride-based omega-3 fatty acid enriching oil to provide DHA, EPA and ALA, for example. That is, phenolic antioxidants and omega-3s can provide complementary and potentially synergistic health benefits if combined together and oxidatively co-stabilized in foods as described herein. This is supported by earlier suggestions of benefits from consuming both phenolic antioxidants and omega-3 fatty acids in ones diet evident in the scientific literature (e.g., as illustrated by results of a web search at <www.ncbi.nlm.nih.gov/sites/entrez> using the search terms, “omega-3” and “polyphenols”). This search suggests use of both these agents in the diet to modulate or control lipoprotein levels, oxidative damage, inflammation, Alzheimer's disease, cancers, inflammatory bowel disease, and cardiovascular disease. A similar search at the same web address using the search terms “grape seed” and “inflammatory” provided additional references. While oxidative decomposition and modes of oxidative stabilization differ for phenolic antioxidants and omega-3 fatty acids, both agents are beneficial to ones health and can be compatibly combined respectively in the aqueous and fat portions of processed foods as described herein.

With regard to the separate health benefits of omega-3 fatty acids, the U.S. FDA has given “qualified health claim” status to eicosapentaenoic acid (EPA) and docosahexaenoic acid (DHA) omega-3 fatty acids for reducing the risk of coronary heart disease (CHD). Concerning additional major benefits provided by omega-3s, fish oil appears to stimulate circulation, promotes fibrin/blood clot breakdown, and decreases blood pressure in some individuals, along with generally decreasing blood triglyceride levels, a risk factor in CHD and heart attacks. EPA can also significant decrease/improve the thickness of carotid arteries along with improvement in blood flow. Moderate levels of EPA and DHA (typically 1-4 g per day) also tend to help reduce cardiac arrhythmias, the incidence of ischemic and thrombotic stroke, as well as the effects of arthritis. Preliminary evidence suggests that EPA and DHA may reduce psychological depression, anxiety aggression and attention-deficit hyperactivity disorder. Several studies also report possible anti-cancer effects of omega-3 fatty acids (breast, colon and prostate cancer).

Still another study with fish oil published in 2007 showed that infants receiving either cow's milk or infant formula supplemented with fish oil showed healthy immune system activation with improved immune function maturation. Research in 2005 and 2006 has suggested that in-vitro anti-inflammatory activity of omega-3 fatty acids translates into clinical benefits. For example, neck pain patients and rheumatoid arthritis sufferers have demonstrated benefits comparable to those receiving standard non-steroidal anti-inflammatory drugs. Other diseases for which amelioration has been reported with the regular consumption of EPA and DHA include Alzheimer's disease, Parkinson's disease, and atopic dermatitis.

While the dietary consumption of natural phenolic antioxidants extracted from fruits and vegetables may provide multiple health benefits, the addition of phenolic antioxidants to commercially processed foods has been limited for a variety of reasons. In addition to the cost of these antioxidant ingredients, their susceptibility to premature oxidation, their astringent taste, and their deep color tend to complicate the use of phenolic antioxidants in many processed foods. Furthermore, their stability in an acidic food environment, but not in a neutral or alkaline pH environment has tended to limit the foods that can be supplemented. The present invention facilitates the addition of phenolic antioxidants to certain types of processed foods, i.e., fat-containing foods, as well as improving the stability and shelf life of phenolic antioxidants in foods.

Phenolic antioxidants as described herein are typically water-soluble chemical compounds, many of which are stable at low pH, allowing their incorporation into acidic food products. Thus, fruit juices, fruit sauces, and other fruit products, as well as tomato-based products, fermented dairy products (e.g., yogurt), and vinegar-containing products (e.g., sauerkraut, soy sauce, mustard, salad dressing) can provide a sufficiently acidic environment for stabilizing phenolic antioxidants. More specifically, these foods typically contain one or more organic acids, e.g., tartaric, maleic, succinic, quinic, citric, acetic and lactic acids that can, at least, partially stabilize phenolic antioxidants and extend the shelf life of the food. In some instances, a sacrificial antioxidant that is more susceptible to oxidation than the phenolic antioxidant is also added (e.g., vitamin C added to grape juice). Utilization of an acidic aqueous environment is described, for example, in the Graff & Hrncirik, Draijer et al., and Zhang applications discussed briefly in the Background, each of which is incorporated herein by reference in its entirety.

In the absence of an acidic environment, phenolic antioxidants can be very unstable and susceptible to both oxidation and hydrolysis, e.g., at neutral and alkaline pH. Without being limited to this mechanism, Applicant believes that this instability may begin with dissociation of the hydroxyl hydrogen in the phenol moiety of the antioxidant molecule. This dissociation, producing the negatively charged phenoxide ion, is favored at neutral to alkaline pH, and results in a chemically reactive molecule that is more susceptible to oxidation. It is interesting to note that aqueous phenol, a toxic laboratory reagent, is also susceptible to alkaline conditions, and is best stored under slightly acidic conditions as described by Perlman in U.S. Pat. No. 5,098,603.

Phenolic antioxidants include, but are not limited to, the monomeric single ring phenolic compounds, e.g., benzoic and cinnamic acid derivatives such as gallic and coumaric acids, and the polyphenolic compounds such as the two ring stilbene derivatives, e.g., resveratrol, the three ring compounds including the flavonoid derivatives such as the flavanols, flavonols, and anthocyanidins. While many of these compounds are present in fruits and vegetables, the catechins including epicatechin (EC), epicatechin gallate (ECG), epigallocatechin (EGC) and epigallocatechin gallate (EGCG) are well known flavonoids (flavan-3-ols) present in teas. The most abundant catechin in tea, EGCG, may constitute as much as 10% of the dry weight of fresh Camellia tea leaves. Accordingly, tea extracts as well as fruit-derived extracts, e.g., grape seed extracts, can be used herein to supplement processed food products. Further, ellagitannins, or punicosides such as ones from pomegranates, e.g., punicalagins, can be beneficially included.

Research in this area is interesting because it is thought that the methods described can have unexpected relevance to the present invention. For example, Ekanayake et al. in U.S. Pat. No. 5,427,806; U.S. Pat. No. 6,268,009; U.S. Pat. No. 6,063,428; and U.S. Pat. No. 5,879,733 describe the processing of green tea extract that initially contains high levels of unoxidized monomeric catechins, epicatechins, epigallocatechins and gallate derivatives. These phenolics are unfortunately easily oxidized to form diverse polymers and complexes with other soluble substances in the extract to produce an undesirable brown color, cloudiness, precipitates and altered taste. Dissolved metal ions, as catalysts, and oxygen in the tea extract aggravate this problem. Ekanayake et al. taught an improved tea extract prepared by extracting the tea with an aqueous acid such as ascorbic plus citric acid, removing the metal cations from the tea extract using a cation exchanger, and passing the extract through a nanofiltration membrane.

While in many cases it will be desirable to utilize plant extracts or plant preparations with substantial and varied phenolic antioxidant content as discussed above, in some cases it may be desirable to utilize single phenolic antioxidants or combinations (which may be artificially created) of different phenolic antioxidant compounds, or combinations in which one phenolic antioxidant compound is present in significantly higher concentration than in plant extracts which have not been enriched or purified for that compound.

Examples of compounds which can be utilized in this way include the substantially water soluble and fat insoluble compounds from among the following, some of which have been mentioned previously:

| | | |
|---|---|---|
| Catechin | coutaric acid | sinapic acid |
| Gallocatechin | fertaric acid | ferulic acid |
| Epicatechin | p-coumaric acid | vanillic acid |
| Epigallocatechin | m-coumaric acid | syringic acid |
| epigallocatechin | o-coumaric acid | p-hydroxybenzoic acid |
| gallate | | |
| catechin gallate | resveratrol | protocatechuic acid |
| | (t-, c-, & mix) | |
| epicatechin gallate | Myricetin | gentisic acid |
| Gallocatechin gallate | myricetin glycosides | hydroxycaffeic acid |
| epicatechin | Quercetin | 3,4-dimethoxy- |
| digallate | | cinnamic acid |
| epigallocatechin digallate | quercetin glycosides | 3,4-dihyroxybenzoic |
| | | acid |
| chlorogenic acid | delphinidin | 4-hydroxycinnamic |
| | | acid |
| Gallic acid | delphinidin | 4-hydroxycinnamoyl- |
| | di-glucoside | quinic acid |
| Caftaric acid | Malvidin | piceatannol |
| Cichoric acid | malvidin di-glucoside | Apigenin |
| caffeic acid | resveratrol glucoside | kaempferol |
| ellagic acid | Peonidin | Luteolin |
| Petunidin | pelargonidin | carvacrol |
| Scopoletin | Apigenin | rhamnetin |
| Eugenol | Capsaicin | hesperidin |
| Isquercitrin | Rutin | Vicenin |
| rosmarinic acid | carnosic acid | Hispidulin |
| Santin | Eupafolin | scutellarein |
| Genkwanin | Acacetin | cirsimaritin |
| Epirosmanol | Rosmanol | rosmarinic acid |
| labiatic acid | Isoetin | chrysoeriol |
| Curcumin | eriodicyoyl | naringenin |
| punicalagins | | |

Thus, the invention includes the use of the above-listed compounds as single purified compounds, in combinations enriched in the particular compound, and in artificial combinations of the listed compounds. Such artificial combinations expressly include each and every combination of the listed compounds taken any 2, 3, 4, 5, 6, 7, 8, 9, 10, 11, or 12 at a time. The listing above includes aglycone forms, as well as glucoside and other glycoside forms and combinations of aglycone and glycosidic forms, whether or not each form is expressly shown in the list.

In some cases, compounds from certain advantageous categories are utilized, e.g., anthocyanidins, anthocyanins, procyanidins, proanthocyanidins, oligomeric proanthocyanidins, and/or oligomeric procyanidins are used.

Examples include homo- and hetero-dimers, trimers, tetramers, pentamers, hexamers, heptamers, and octamers of catechin (C), gallocatechin (GC), epicatechin (GC), epigallocatechin (EGC), epigallocatechin gallate (EGCG), catechin gallate (CG), epicatechin gallate (ECG), and gallocatechin gallate (GCG). Examples of such heteropolymeric forms include ECG+C, ECG+EC, ECG+2C, ECG+2EC, 2EGCG+C, 2EGCG+EC, ECG+3C, ECG+3EC, ECG+4C, ECG+4EC, ECG+5C, ECG+5EC, ECG+6C, ECG+6EC, ECG+7C, and ECG+7EC, which may be used singly or in any combination.

### Solution for Omega-3-Associated Off-Flavor Development

As indicated above, in response to the growing awareness that omega-3 fatty acids can provide substantial health benefits to humans of all ages, a number food producers have begun adding omega-3 fatty acids to foods, e.g., supplementing conventional cow's milk products with flaxseed oil, providing alpha-linolenic acid (ALA) and/or fish oil (providing EPA and DHA). It has been observed that off-flavor development in such omega-3-supplemented milks can occur, and sometimes (e.g., during the summer season) it is a regular problem during the shipping and storage of these milks (as well as other food products). Off-flavor development has been characterized as a somewhat “fishy” flavor, or other unexpected flavor. Such off-flavors are reported more frequently with skim milk and 1% milks than with higher milkfat-content products. Indeed, the oxidative stability problem of fish oil in milk has been recognized for years, and only limited progress has been made in solving this essential problem that involves complex chemistry.

As a remedy for the instability of the omega-3 fatty acids in milks and other food products, it has been found that it is beneficial to dissolve the unstable omega-3-fatty acid-containing fish oil in an “oxidative stabilization oil,” i.e., a carrier fat or oil such as an oxidation-resistant vegetable oil. The carrier oil used is advantageously substantially more resistant to oxidation than the omega-3 fatty acid-containing oil. In certain advantageous cases, the carrier oil (that acts as a chemical diluent for the omega-3 fatty acid enriching oil, e.g., fish oil) is an oil high in monounsaturated and/or saturated fatty acids and low in polyunsaturated fatty acids (e.g., preferably no more than about 20% polyunsaturated fatty acids). It is especially preferable that the carrier oil is low in linoleic acid. Particularly preferably as the carrier oil is a high-oleic, low-linoleic fat or vegetable oil. One example of such a carrier oil is high oleic/low linoleic acid sunflower oil (e.g., Clear Valley Sunflower Oil or Odyssey 100 Sunflower Oil sold by Cargill, Inc. (Minneapolis, Minn.) containing 10% saturated fatty acids, 82% by weight monounsaturated oleic acid and only 8% linoleic acid.

Also useful as carrier oils, particularly in liquid food applications where the oil droplet or particle size is reduced by homogenization or emulsification, are certain oils and fats that contain high levels of saturated fatty acids and low (or exceptionally low) levels of polyunsaturated fatty acids, e.g., palm oil (PO), palm kernel oil (PKO), and palm kernel stearin (PKS). For example, PKS (product # CLSP 499 obtained from Loders Croklaan, Channahon, Ill.) contains as much as 92% saturated fatty acids, 7% monounsaturated fatty acids, and only 1% polyunsaturated fatty acids. PKS has the desirable mouth feel property of being liquid at body/mouth temperature in spite of being solid at room temperature. Despite these preferences, a variety of different oils and oil blends may be used which have substantially greater oxidative stability as compared to omega-3 fatty acid-containing oils.

It is preferable to dissolve one part by weight of an omega-3-enriching oil (e.g., EPA/DHA enriching oil) such as a fish oil and/or flax seed oil in at least two parts by weight of an oxidative stabilization oil to achieve at least a 3-fold dilution of the omega-3 fatty acids relative to their original concentration in the enriching oil. In theory, a 3-fold dilution of the omega-3 fatty acids could reduce the rate of omega-3 oxidation up to 9-fold. Of course, lower dilutions can be used, with corresponding lower levels of omega-3 fatty acid stabilization expected.

Therefore, to provide a 3-fold dilution, if 100 mg of fish oil is to be added as a supplement to a serving of a food product, it can first be diluted with at least 200 mg of oxidative stabilization oil such as the low linoleic/high oleic-containing sunflower oil described above. Greater dilutions of the omega-3-enriching oil are even more preferred, with, for example, 300-500 mg sunflower oil being used as the oxidative stabilization oil for 100 mg of fish oil to provide a four to six-fold dilution rather than a 3-fold dilution of the omega-3 (e.g., EPA/DHA) enriching oil.

The resulting mixture or blend of omega-3 enriching oil and omega-3 stabilization oil (i.e., an oxidative stabilization oil) that is added and blended (e.g., homogenized) in a food product may be conveniently referred to as an “omega-3 fatty acid-containing supplementation oil”, or simply as a “supplementation oil”.

Though the low linoleic/high oleic oil is preferred for the oxidative stabilization oil, other fats and/or oils may be used, e.g., cocoa butter, palm fat, palm stearin, conventional palm oil, palm olein, palm superolein, and palm kernel oil (the palm oil and derivatives being low linoleic (e.g., about 9-11%)/high saturated fat oils), as well as conventional canola oil, soybean oil, cottonseed oil, corn oil, sunflower oil, milk fat, milkfat fraction, and/or safflower oil, as well as combinations of such oils.

When an oxidative stabilization oil is used which solidifies at normal processing temperatures, it is beneficial for the blend of omega-3 enriching oil and oxidative stabilization oil to be formed at temperatures at which the stabilization oil is liquid. The blended oil can then be solidified by cooling, resulting in a low molecular mobility for triglycerides containing the omega-3 and other polyunsaturated fatty acids.

In forming the blend of omega-3 enriching oil and omega-3 oxidative stabilization oil, in many cases, a single stabilization oil will be used. However, as indicated above, more than one oil may be used in combination as an oxidative stabilization oil. Such a combination will often be formed by mixing more than one oil to form the oxidative stabilization oil, before blending with the omega-3 enriching oil. However, the blend may also be formed by combining more than one oil, which together act as an oxidative stabilization oil, with the omega-3 enriching oil without premixing or with only partial premixing of the components of the oxidative stabilization oil. In many embodiments, the various oil components of the oxidative stabilization oil will each be oxidative stabilization oils, but alternatively, one or more of those component oils will not be oxidative stabilization oils alone, but the combination is an oxidative stabilization oil.

### Inclusion of Antioxidants in Oil Phase of Food Compositions

As an approach to enhance the oxidative stabilization effects of dilution of omega-3 fatty acid-containing oils by dilution in an oxidative stabilization oil, or as an alternative to that approach, fat/oil soluble, water insoluble antioxidants can be included in the food composition, and especially in compositions having both aqueous phase and oil phase, e.g., mayonnaise and salad dressings containing both vinegar and oil. In this approach, at least one such antioxidant is blended with an omega-3 fatty acid-containing edible oil, and/or with an oxidative stabilization oil which is simultaneously or subsequently mixed with an omega-3 fatty acid-containing oil.

Using antioxidants to protect omega-3 fatty acids and other polyunsaturated fatty acids against oxidation in food products and/or food product components involves selection of appropriate antioxidants. The antioxidants should be fat/oil soluble, water insoluble antioxidants, or be antioxidants which can be used at sufficiently high concentrations and having sufficiently low solubility in water so that the residual antioxidant concentration in the oil phase of the food product is still sufficiently high so as to provide effective antioxidant protection. A number of antioxidant compounds are commonly used in foods. These include, for example, TBHQ, BHA, and BHT.

Tert-butylhydroquinone (TBHQ), also identified as 2-(1,1-Dimethylethyl)-1,4-benzenediol, is used as a food preservative, including as an antioxidant in edible oils. It is currently regarded as the most effective antioxidant for such oils and is stated to be effective in foods (e.g., fried foods) prepared using such oils. Nonetheless, TBHQ is less desirable for use as an antioxidant in the present invention because it has appreciable water solubility. As a result, even if initially present in the oil phase of the emulsion, it will rapidly partition between the oil and aqueous phases. This can result in a substantial reduction in the concentration in the oil phase.

On the other hand, BHA (butylated hydroxyanisole) and BHT (butylated hydroxytoluene) have sufficiently sparing solubility in water that only a small amount of these compounds will partition from the oil phase to the water phase. As a result, inclusion of one or both of these compounds in an oil preparation as indicated above, which is then mixed and homogenized with aqueous phase, will provide effective oxidation protection.

Vitamin E (e.g., as D-alpha-tocopherol or D,L-alpha tocopherol) can also be added, and can serve as an antioxidant for the oils in an oil:water suspension. Vitamin E can also be added as a dietary supplement (most often in the form of D- or D,L-alpha-tocopheryl acetate), e.g., at levels of about 0.01 to 0.02% by weight of the aqueous suspension. For use as an antioxidant for the oil in contact with an aqueous solution or suspension, an active form (e.g., free tocopherol) is added to the oil, in many cases at a level of about 100 to 5000 ppm or more commonly about 200 to 2000 ppm in the oil, e.g., about 200 to 500, 300 to 700, 500 to 1000, 700 to 1500, or 1000 to 2000 ppm. Other isomers of tocopherol can also be used as alternatives or in addition, such as beta-tocopherol, gamma-tocopherol, delta-tocopherol, and combinations thereof.

In addition, the inclusion of phenolic antioxidants in an aqueous acetic acid component of a food can be further combined with inclusion of functional levels of microparticulate oil-insoluble antioxidants in a stabilizing oil, e.g., as described in Perlman, U.S. patent application Ser. No. 12/372,773, entitled Stabilization of Phenolic Antioxidants in Fat-Containing Foods, which is incorporated herein by reference in its entirety.

As described therein, of the many edible fruit and vegetable sources of phenolic antioxidants, a number of current exemplary sources that are both concentrated and cost-effective for use in processed foods include: (a) a microparticulate purified grape seed extract that contains at least 90% by weight phenolic antioxidants (e.g., ActiVin®), (b) microparticulate purified green tea extracts that contain high levels of catechin-type phenolic antioxidants from Camellia sinensis leaves, (c) microparticulate purified pomegranate extracts that contain high levels of punicalagin-type phenolic antioxidants from the pomace of the pomegranate fruit, and (d) microparticles of milled grape seed flour from cold-pressed viniferous grapes (containing insoluble fiber and water soluble phenolic antioxidants, usually up to approximately 10% water-soluble phenolic antioxidants). Applicant hypothesized that phenolic antioxidants contained in these microparticles might be degraded via oxidation more slowly in a fat environment rather than in water. However, one line of reasoning suggested that fat might accelerate rather than reduce the rate of oxidation of phenolics occurring in water.

More specifically, although phenolics tend to be insoluble in vegetable oil, the molecular oxygen component in air at room temperature and one atmosphere pressure is approximately five times more soluble in vegetable oils, e.g., soybean oil, than in water. This raised the possibility that fat/oil-borne oxygen might accelerate the decomposition of phenolic antioxidants particularly during heat processing of fatty foods. On the other hand, vegetable oil (e.g., soybean oil) has an intrinsic viscosity that is approximately fifty times greater than that of water at room temperature. This increased viscosity might reduce the amount of molecular oxygen reaching phenolic antioxidants suspended in oil versus water, and thereby reduce the reaction rate between oxygen and the phenolic compounds. Accordingly, microparticulate ActiVin® grape seed extract was used in a series of experiments in which Applicant measured and compared the levels of phenolic antioxidant surviving in a vegetable oil medium compared to several edible aqueous media. Results demonstrated that the oil medium provided a stabilizing environment for the phenolic antioxidants.

As also described in Perlman, U.S. patent application Ser. No. 12/372,773, the oil can shield the phenolic antioxidants such that less of the astringent taste normally associated with phenolic antioxidants is perceived upon ingestion. Thus, in conjunction with the reduction of astringency for phenolic antioxidants in aqueous acetic acid in foods, the use of phenolic antioxidants stabilized in oils in the same foods allows substantial levels of such phenolic antioxidants to be added to such foods without excessive astringency.

Phenolic antioxidant compounds such as catechins and proanthocyanidins are well known for their astringency, particularly when present in foods and beverages at levels ranging from approximately 50-500 mg phenolics per serving (0.02%-0.5% by weight phenolics for 3 to 8 ounce serving sizes). The perception of astringency (also described as “mouth puckering”) depends upon the interaction between sensory receptors in the mouth and solubilized phenolic compounds. In the present invention, the solubilization of most phenolics is prevented or retarded by the presence of fat that bathes the microparticles carrying phenolic antioxidants. For example, when grape seed flour or grape seed extract particles are added to peanut butter or margarine and coated with fat before the food is tasted, the water-soluble phenolic antioxidants that would otherwise mix with saliva and taste as astringent, are partially masked by the fat. In the case of finely milled grape seed flour (e.g., 100-140 mesh size), the phenolic compounds are more sequestered within the flour particles and less prone to being tasted as astringent than with purified grape seed extract particles.

More specifically, approximately 90% by weight of the grape seed flour microparticle is non-phenolic material (fiber, protein, carbohydrates), that can substantially mask the taste of the 10% by weight phenolics. Furthermore, the phenolics are slow to diffuse from these oil-coated flour microparticles. On the other hand, the astringency from phenolics contained in microparticles of purified grape seed extract (e.g., ActiVin® microparticles typically containing 90% by weight or more of water-soluble phenolic antioxidants) is less well masked by combining with a fatty food such as peanut butter. This difference is attributable to the more rapid solubilization of phenolics contained in these extract microparticles compared to grape seed flour microparticles when mixed with saliva in the mouth. It is expected that the use of solid digestible solid material microparticles such as solid fat or wax microparticles will provide effective reduction of perceived astringency with both purified phenolic antioxidants and with plant material flour microparticles (or other fibrous microparticles) containing phenolic antioxidants.

Nevertheless, this method of using a fat or a fatty food as a carrier or vehicle for microparticles that contain substantial phenolic antioxidants, e.g., between 5% and 99% by weight phenolic antioxidants [percentage by weight phenolics measured as gallic acid equivalent (GAE) percentage], is an effective means of counteracting the astringency contributed by phenolic antioxidants added to fats and fatty foods. The mesh size of microparticles is preferably smaller than 80 mesh, e.g., 100 mesh (0.006 inch or 150 microns), and more preferably 140 mesh or smaller (0.004 inch or 100 microns) or even 200 mesh or smaller (0.003 inch or 75 microns). The ActiVin® microparticulate material obtained from San Joaquin Valley Concentrates, Inc. is approximately 50 microns in size.

Astringency Test for Phenolic Antioxidants Added to Water vs. Acetic Acid Solution

A taste test of phenolic antioxidants in water, sodium acetate solution, and acetic acid solution was carried out. In all cases, a 0.2% by weight solution of ActiVin® microparticulate material (from San Joaquin Valley Concentrates, Inc) in water was used. To aliquots of this solution were added acetic acid, NaAcetate, citric acid (another edible acid), or NaCitrate (another edible salt) in amounts as shown in the table below.

| Added compound | Molarity (wt %) | pH | Perceived Taste |
|---|---|---|---|
| None | — | Approx 7 | Highly astringent |
| Acetic acid | 0.1M (0.6%) | 2.9 | Moderate bitterness |
| | 0.4M (2.4%) | | Slight astringency; |
| | | | slight sweetness |
| | 0.8M (4.8%) | | No astringency; slight |
| | | | sweetness |
| NaAcetate | 0.1M (0.8%) | 8.0 | Reduced astringency; |
| | | | slight salty taste |
| | 0.4M (3.2%) | | Reduced astringency; |
| | | | salty |
| Citric acid | 0.1M (1.9%) | 2.2 | Elevated astringency |
| | | | (more than in water |
| | | | only) |
| | 0.4M (7.6%) | | Extreme astringency |
| | | | (not tolerable) |
| NaCitrate | 0.1M (2.6%) | 8 | Astringent; salty |
| | 0.4M (10.4%) | | Astringent; very salty |

As the taste results show, solutions containing acetate ion were more effective than either citric acid or NaCitrate in reducing or eliminating phenolic antioxidant astringency. Further, the acetic acid was significantly more effective than the NaAcetate in reducing astringency and providing an overall flavor improvement. The citric acid/citrate results indicate that mere acidity is insufficient to provide the astringency reduction; that the acetic acid/acetate has distinctively better and surprisingly advantageous effects.

### Definitions

To assist the understanding of the reader, in discussing the present invention and in the claims, the following terms are applicable and have the indicated meanings.

The term “food or beverage composition” within the context of the present invention refers to any composition which is suitable for human consumption.

As used herein, the term “acetic acid solution” refers to a solution with acetic acid in water.

The term “vinegar” refers to an acetic acid solution which contains from 1 to 10 percent by weight acetic acid and more commonly about 4 to 8 percent, and which may contain flavor and/or other types of compounds. Examples include glacial vinegar, apple cider vinegar, balsamic vinegar, and the like.

The term “aqueous suspension” refers to a suspension of one or more species in water. Such species may include, for example, proteins and/or oils. In many but not all cases, such an “aqueous suspension” will be an “emulsion”.

The two terms, “phenolic antioxidants” and “polyphenolic antioxidants,” and the measured concentrations thereof, refer to the collective population of molecular species made by plants (and ingested by animals) containing one or more aromatic ring structures having at least one hydroxyl substituent. For the purposes of this invention, these two terms are used interchangeably unless a distinction is made clear.

In the context of additions of phenolic antioxidants to aqueous solutions or suspensions and/or edible oils or other edible oil-containing food compositions, the terms “supplementary”, “exogenous”, “exogenously added” and like terms means that the phenolic antioxidants are added to a food composition by people, as distinguished from phenolic antioxidants that are naturally present. Thus, for example, phenolic antioxidants in the form of grape seed flour and/or grape seed extract which are added to an oil (which could be a grape seed oil) or another food composition are “exogenous” or “exogenously added” phenolic antioxidants, while phenolic antioxidants which are found in olive oil or grape seed oil obtained by cold pressing are “endogenous” phenolic antioxidants and are not “exogenously added.”

For the purposes herein, the concentration or “percentage by weight” of phenolic or polyphenolic antioxidant is assayed and expressed as an equivalency to a percentage by weight of gallic acid; i.e., gallic acid equivalents or GAE units that are units of concentration. These so-called phenolic or polyphenolic concentrations are measured using a colorimetric assay based upon reacting phenolic/polyphenolic compounds with Folin-Ciocalteau reagent (abbreviated “F-C reagent”). This assay of phenolic chemical groups does not distinguish between simple phenolic derivative compounds and more complex polyphenolic structures. For the purposes herein, phenolic antioxidants represent all of the phenolic group molecular species (molecular structures) that remain soluble in an aqueous liquid such as a beverage or water-containing food such as a soup, condiment, aqueous emulsion, bakery product and the like. Phenolic and polyphenolic antioxidants can include some molecules that have already undergone a limited amount of oxidation and/or polymerization due to exposure to air, light.

In the Folin-Ciocalteau assay, a gallic acid standard solution (1.00 mg/ml) is used to generate a linear standard curve. Increasing amounts of the gallic acid solution (between 2.5 and 15 microliters) are diluted into a series of sample test tubes holding 0.50 ml water. Next, 50 microliters of F-C reagent (Sigma Chemical Company) is added to each tube. After 1 minute, but before 8 minutes following addition of the F-C reagent, 0.25 ml of a 15% by weight aqueous sodium carbonate solution is added, the samples are vortexed, and then incubated (maintained) for 2 hours at room temperature. The optical absorbance at 760 nm is read. A sample that is constituted with all chemical components but without gallic acid is also incubated as used as a blank sample to zero the sprectrophotometer (Spectronic 20D+ manufactured by Thermoelectron Corp.). This blank registered an absorbance (optical density or O.D.) at 760 nm of approximately 0.005 above that of distilled water. In the assay, an O.D. 760 nm reading of 1.3-1.4 corresponded to approximately 10 microliters of 1.00 mg/ml gallic acid. Also, for reference purposes, a commercial single strength Concord 100% grape juice (Welch's) was shown to have the equivalency in the F-C assay of approximately 0.25% gallic acid (0.25 GAE units).

As antioxidants, the phenolics can scavenge unpaired electrons (free radicals), inactivate reactive oxygen species, and chelate metal ions that catalyze oxidation. A partial list of prevalent phenolic species include the simple cinnamic and benzoic acid derivatives, the stilbenes (2 phenolic rings), the 3 ring flavonoids (2 phenolic rings plus a flavone ring) that include catechins, flavanols, the anthocyanidins (not glycosylated) and the positively charged anthocyanins of many different structures (glycosylated anthocyanidins having colors ranging from red to blue), and the four ring ellagic acid species and its derivatives as well as a variety of tannins, to name a few.

The term “astringency” as used herein is the taste sensation or mouth feel that is most apparent as an aftertaste, and is often described as mouth puckering. Astringency is often associated with the tannin content of immature wines, i.e., wines that are not sufficiently aged. The sensation of astringency is thought to be caused by a reaction between phenolic compounds such as the tannins and the so-called PRP proteins (proline-rich proteins) in saliva that are thought to provide wetting, lubrication and protection of the oral epithelium. Research suggests that the precipitation and/or aggregation of complexes formed between the salivary proteins and phenols results in loss of oral lubricity-thus the tightened, dry, rough or “puckery” sensation on oral surfaces such as along the sides of the taster's tongue.

The term “sacrificial antioxidant” refers to a chemical substance that is added to a processed food composition for the purpose of protecting an ingredient that is susceptible to oxidation. By being more susceptible to oxidation than the ingredient being protected, the sacrificial antioxidant is consumed first before an appreciable amount of the valuable ingredient is lost. Examples of these sacrificial antioxidants include vitamin C, rosemary extract, TBHQ, BHA, BHT, propyl gallate and combinations and derivatives thereof that are edible food additives and GRAS (see above) at the levels prescribed by governmental regulations.

The term “shelf life” or “shelf-stable” in the context of phenolic antioxidants contained in a processed food product refers to a loss of less than 25% per year in the phenolic antioxidant content of the material when stored at 20° C.

In connection with the level of phenolic antioxidants in the present food products and methods, a “water-astringent level” refers to a concentration of phenolic antioxidants dispersed in an aqueous acetic acid solution or suspension which is unacceptably astringent for commercial food use when dissolved in an aqueous solution or suspension which is the same except for the absence of the acetic acid, when the solutions or suspension are subjected to controlled taste tests by experienced taste testers having normal perception of astringency.

Also in connection with the level of phenolic antioxidants in the present food products and methods, a “non-astringent level” of the phenolic antioxidants refers to a concentration of the phenolic antioxidants which are perceived as substantially non-astringent when the solutions or suspension are subjected to controlled taste tests by experienced taste testers having normal perception of astringency.

As used in reference to blending of oil with an aqueous solution or suspension, the term “homogenized” refers to the blending of the components with high shear mixing or other effective blending method, by which an edible oil (or traditionally cream) is uniformly and stably dispersed into the aqueous component (or the converse) so that the edible oil (in the form of micro-droplets) does not substantially separate from the bulk of the aqueous suspension and float to the top (or the converse). Highly preferably there will be no substantial separation over the normal shelf life for the resulting product.

The terms “EPA/DHA fatty acid-containing enriching oil” and “EPA/DHA fatty acid-containing oil” refers to any edible oil that is predominantly triglyceride-based and contains an abundance of the omega-3 fatty acids, EPA and/or DHA. The term “abundance” as used herein means that the edible oil contains at least a total of 10% by weight EPA+DHA fatty acids, and preferably 20-35% or even 35-60%, or higher EPA+DHA fatty acids.

The terms “alpha-linolenic fatty acid-containing enriching oil” and “alpha-linolenic acid-containing oil” refer to any edible oil that is predominantly triglyceride-based and contains an abundance of the omega-3 fatty acid, alpha-linolenic acid (abbreviated ALA). The term “abundance” when used with ALA means that the edible oil contains at least 25% by weight ALA and preferably 35% by weight or more ALA.

Similarly, the terms “omega-3 enriching oil” and “omega-3 fatty acid-containing enriching oil” and like terms refer to an edible oil that is either or both of an “EPA/DHA fatty acid-containing enriching oil” or an “alpha-linolenic fatty acid-containing enriching oil”.

Further distinguishing the present omega-3 fatty acid-containing supplementation oils from conventional cooking and salad oils is that a substantial proportion of the triglyceride molecules in the supplementation oils contain two, and sometimes three, omega-3 fatty acids esterified within the same triglyceride molecule. Thus, for the three glycerol carbon positions within omega-3-containing triglyceride molecules found in the supplementation oils, often the sn-1 and sn-2, or the sn-2 and sn-3, or the sn-1 and sn-3 positions are esterified with omega-3 fatty acids.

The terms “omega-3 fatty acid-containing supplementation oil”, “supplementation oil”, and like terms such as those containing reference to an aqueous suspension or oil-water suspension (e.g., “omega-3 fatty acid-containing supplementation oil”) are used to refer to an edible oil composition that includes omega-3 fatty acids along with other fatty acids in proportions such that the rate of oxidation of the omega-3 fatty acids is significantly reduced as compared to the rate of oxidation of the omega-3 fatty acids in a conventional cod liver oil containing at least 30% by weight of a combination EPA and DHA. Such oxidation rate is determined for oils (or oil-containing food product) held at 4 degrees C. with air exposure of at least 50 cm² per liter. The significant reduction is a statistically significant reduction, preferably such that the rate of oxidation in the supplementation oil is not more than 0.80, 0.70, 0.50, 0.30, 0.20, 0.10, 0.05, 0.02, 0.01, or 0.005 of the rate in the cod liver oil. In many advantageous cases, the supplementation oil is a blended oil composition, i.e., a mixture of edible oils, that includes:

(a) an omega-3 fatty acid-containing enriching oil ((providing EPA and/or DHA and/or ALA, see above) that is susceptible to oxidation and, that is combined and diluted with

(b) a triglyceride-based edible oil that possesses good oxidative stability compared to the oxidative stability of oils high in omega-3 fatty acids. Preferably such oil is low in polyunsaturated fatty acids (especially linoleic acid) and high in monounsaturated (e.g., oleic) and/or saturated fatty acids. Preferred examples of the edible oil having good oxidative stability can be referred to as “oxidative stabilization oils”, such as low linoleic/high oleic sunflower oil.

Thus, the term “oxidative stabilization oil” refers to a triglyceride-based edible oil that is substantially more resistant to oxidation than EPA and/or DHA fatty acid-containing enriching oils. Such oxidative stabilization oil preferably contains less than 20% and more preferably less than 17% 15%, 12%, 11%, 10%, 9%, or 8% by weight polyunsaturated fatty acids or specifically linoleic acid. Preferably such oxidative stabilization oil also contains more than 65% and preferably more than 70%, 75%, or 80% by weight monounsaturated and/or saturated fatty acids. In desirable embodiments, the low linoleic acid content oxidative stabilization oil is either a high oleic acid content vegetable oil, or a saturated fat or oil that contains high levels of one or more saturated fatty acids (including lauric, myristic, palmitic, and stearic acid and combinations thereof) as well as such combinations also including oleic acid. Thus for example, high oleic sunflower oil sold as Clear Valley® High Oleic Sunflower Oil or Odyssey® 100 High Stability Sunflower Oil produced by Cargill, Inc. (Minneapolis, Minn.) contains only 8% linoleic acid, 8% palmitic+stearic saturated fatty acids, and 82% monounsaturated oleic acid, and a highly saturated palm kernel oil (PKO) or palm kernel stearin (PKS) may contain as little as 1% linoleic acid, 7% monounsaturated oleic acid and 92% saturated fatty acids of which lauric and myristic acids are the predominant fatty acids. Advantageously, oxidative stabilization oils preferably contain no more than 15% by weight linolenic acid (generally as ALA) and more preferably no more than 12, 11, 10, 9, 8, 7, 6, 5, 4, 3, 2, or 1% by weight, and/or no more than 2% EPA+DHA, and more preferably no more than 1.5, 1, 0.7, 0.5, 0.2, or 0.1%.

The term “fish oil” is discussed elsewhere herein. Fish oil is refined from the tissues of many varieties of oily fish such as mackerel, sardines and herring. Fish oil commonly contains between 20% and 30% by weight of a combination of EPA and DHA long chain polyunsaturated fatty acids. The fish do not actually produce omega-3 fatty acids, but instead accumulate them by consuming microalgae (also termed “algae” herein) that produce these fatty acids or other organisms which have accumulated those fatty acids. Marine microalgae, or phytoplankton, provide the food base for the entire sea animal population. The best known microalgae are the diatoms, dinoflagellates, green algae and blue-green algae. These microalgae species produce a wide range of lipid fatty acids including significant quantities of the essential polyunsaturated fatty acids, linoleic acid, alpha-linolenic acid and the highly polyunsaturated omega-3 fatty acids, octadecatetraenoic acid (C18:4), eicosapentaenoic acid (C20:5) and docosahexaenoic acid (C22:6).

Thus, the term “algae oil” or “algal oil” refers to an oil obtained from lipid-producing microorganisms, including for example, diatoms, dinoflagellates, green algae, and/or blue-green algae. Commonly such algae oil is obtained from green algae. Such algae oils which are high in omega-3 fatty acids can be used in the present invention.

The term “interesterified” used within the context of an EPA and DHA fatty acid enriching oil refers to the optional use of enzymatic or chemical cleavage of these fatty acids from the natural triglyceride molecule, followed by esterification, by which the average number of EPA and/or DHA fatty acids esterified (attached by an ester linkage) per fat molecule may be increased. Fish oils so altered by interesterification may contain upward of 50% by weight EPA/DHA.

The term “high oleic” as used herein refers to edible oils containing at least 65% and preferably at least 70%, 75%, or 80% by weight of the monounsaturated fatty acid, oleic acid. Plant breeding has allowed the genetic selection of a variety of high oleic vegetable oil species including but not limited to sunflower oil, safflower oil, canola oil, and soybean oil.

The term “rate of oxidation” in the context of oxidation of EPA and DHA fatty acids within an edible oil that is added to a food product according to the methods described herein, refers to the rate of accumulation of by-products from fatty acid oxidation including acids, aldehydes, and ketones, for example. These by-products are produced by peroxidation or addition of oxygen atoms to the fatty acids contained within fish oil triglyceride molecules. The accumulation of such oxidative by-products may be measured by a variety of methods known to those skilled in the art, including, for example, organoleptic evaluation methods by which rancidity in a food sample becomes detectable by taste and/or smell and chemical, as well as chemical analytical methods.

As used herein in connection with edible oils, the term “artificial mixture” refers to a mixture or blend created by a person or persons of two or more oils from different sources and having different characteristics. Similarly, the terms “artificially blending” and “artificially mixing” refer to a blending carried out by a person or persons.

In reference to inclusion of antioxidant compounds to oils in food products containing aqueous acetic acid solutions or suspensions, the term “effective amount” or an indication that the antioxidant(s) are “effective” means that the antioxidant(s) significantly reduce the rate of oxidation of polyunsaturated fatty acids or particularly of omega-3 fatty acids in the oil as compared to the rate of oxidation with conditions the same except for the absence of the antioxidant(s). Advantageously, in some cases the rate of oxidation is reduced to no more than 95, 93, 90, 80, 70, 60, 50, 40, 30, 20, or 10% of the oxidation rate in the absence of the antioxidant(s).

In connection with the use of antioxidants in the present invention, the term “fat soluble/water insoluble” means that the particular antioxidant compound has a vegetable oil/water partition coefficient at 4 degrees C. (based on an approximately average canola oil) of at least 20, but preferably at least 25, 50, 100, 200, 300, 500, 700, or 1000. In this context, the partition coefficient is the ratio of the concentration of the solute in the vegetable oil to the concentration of the solute in the water at equilibrium (Cₒ/C_{w})

Also in the context of the use of antioxidants in the present invention, the term “fat soluble” indicates that the antioxidant is sufficiently soluble in a present supplementation oil at 4 degrees C. to effectively reduce the rate of oxidation of polyunsaturated fatty acids in that oil, and/or to have a solubility in average canola oil at 4 degrees C. of at least 50, and preferably at least 100 ppm by weight. In some cases, the solubility will be greater, e.g., at least 200, 500, 700 or 1000 ppm.

In reference to a particular type of vegetable oil, the term “average” means that the components (primarily the particular fatty acids) of the oil have median values based on a large number of independent geographically and temporally diverse samples of the specified oil.

In reference to food products, the term “normal serving” refers to the quantity of that food product which matches FDA requirements for serving size definitions for nutritional labeling purposes, e.g., based on FDA-established lists of “Reference Amounts Customarily Consumed Per Eating Occasion.” If the serving size is not defined by such FDA requirements, then the serving size is the amount of that food customarily eaten at one time based on consumer data. In reference to an edible aqueous suspension which is not itself the food product in question, unless indicated to the contrary in the context of a particular food product which incorporates the aqueous suspension the term “normal serving” refers to the quantity of the aqueous suspension incorporated in a “normal serving” of that food product.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made in the particular choice of phenolic antioxidants, aqueous acetic acid solution, oxidative stabilization oil, source of EPA/DHA or alpha-linolenic fatty acid-containing enriching oils, method of combining and diluting edible oils, method of measuring the rate of oxidation of omega-3 fatty acids in food products, and the like. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

1. A reduced astringency phenolic antioxidant supplemented composition suitable for human consumption comprising:
an aqueous component containing at least 0.5% by weight acetic acid, or a molar equivalent amount of edible acetate salt, and supplementary phenolic antioxidants at a level such that the total level of phenolic antioxidants in said aqueous component is at least a water-astringent level, wherein the astringency of the phenolic antioxidants in the aqueous component is reduced as compared to the astringency in the absence of said acetic acid.

2-42. (canceled)

43. A method for making a food product containing stabilized, reduced astringency phenolic antioxidants, comprising:
artificially blending phenolic antioxidants into an aqueous acetic acid or acetate salt component of said food product, wherein the phenolic antioxidants in said aqueous acetic acid or acetate salt component have reduced astringency as compared to the astringency of the phenolic antioxidants in water.

44-48. (canceled)

49. A method for reducing free radicals and/or deleterious compounds formed in a food composition during cooking, comprising:
contacting said food composition with a solution or suspension which comprises aqueous solution containing at least one edible phenolic antioxidant preparation mixed with an edible acetic acid or acetate salt, whereby the presence of said phenolic antioxidants reduces the amount of free radicals and/or deleterious compounds formed during cooking present in said food composition compared to the level in the absence of said solution or suspension.

50-54. (canceled)
